(19) United States
(12) Patent Application Publication
(10) Pub. No.: US 2016/0076075 A1
(43) Pub. Date: Mar. 17, 2016

(54) ENZYMATIC HYDROLYSIS OF GLUCURONIDE CONJUGATED DRUGS IN THE PRESENCE OF WATER MISCIBLE ORGANIC MEDIA

(71) Applicant: Ameritox, Ltd., Baltimore, MD (US)

(72) Inventors: Gregory L. McIntire, Greensboro, NC (US); Ayodele Morris, Midland, TX (US); Scott Almon Chester, Greensboro, NC (US); Erin Strickland, Greensboro, NC (US)

(21) Appl. No.: 14/851,531

(22) Filed: Sep. 11, 2015

Related U.S. Application Data
(60) Provisional application No. 62/048,935, filed on Sep. 11, 2014.

(51) Int. Cl. C12Q 1/34

(52) U.S. Cl. CPC C12Q 1/34; G01N 2333/924; G01N 2430/00

(57) ABSTRACT

The present disclosure provides methods of hydrolyzing a drug-glucuronide conjugate, and methods of determining a drug concentration comprising hydrolyzing a drug-glucuronide conjugate in the presence of a water miscible organic solvent that can simultaneously prevent bacterial growth in the enzyme and prevent analyte adsorption.

### PRIORITY CLAIM

This application claims priority to U.S. Provisional Patent Application Ser. No. 62/048,935, filed Sep. 11, 2014, the entire contents of which are incorporated herein by reference and relied upon.

### FIELD

The present disclosure provides methods of enzymatically hydrolyzing glucuronide conjugated drugs in the presence of a water miscible organic solvent or solvents.

### BACKGROUND

Many drugs are metabolized in the body via conjugation with glucuronic acid (Baselt, 2008). In some cases, such conjugation follows oxidation and/or hydroxylation generally via cytochrome P450 enzymes. In other cases, hydroxyl (—OH) groups resident upon the molecule can be conjugated directly. In either case, drugs are frequently excreted (e.g., in urine) as a glucuronide conjugate. Such conjugates are often unstable under typical analytical conditions (e.g., mass spectroscopy). For example, under commonly used ionization conditions in the source of the mass spectrometer, glucuronide conjugates can be degraded as well as ionized leading to unstable analytical results (Enders et al., 2012). The limited availability of reference standards for conjugates and the desire for improved detection are added factors that compel sample hydrolysis as part of sample processing for the analysis of heavily conjugated compounds (McIntire et al., 2013; Levine, 2010). Cleaving the conjugate before analysis is also frequently problematic; some conditions suitable for hydrolysis also effectively degrade or structurally rearrange the drug itself (Zezulak et al., 1993; Levine, 2010). This can complicate or introduce error into analysis of drug concentration (e.g., for monitoring drug compliance or deviation).

Enzymatic hydrolysis of glucuronide conjugates has also been used to avoid degradation of the drug compound such as observed when strong acid or base is used to cleave the conjugate. Historically, these enzymes have been derived from bacteria, snails or abalone. However, enzymatic hydrolysis using these enzymes typically requires long reaction times and heat to achieve complete reaction (Feng, et al. 2001; Miki et al., 2002). In addition, the conjugate and the cleaved glucuronic acid are often much more polar than the cleaved drug, which introduces complications in processing due to differential solubility.

Analysis (including rapid screening and high throughput processing) of drugs excreted as glucuronide conjugates has therefore been riddled with complications and potential sources of error.

New and improved methods for analyzing drugs excreted as glucuronide conjugates are needed.

### SUMMARY

In various embodiments, the present disclosure provides methods for enzymatically hydrolyzing a glucuronide conjugated drug in the presence of a water miscible organic solvent or solvents. In some embodiments, the organic solvent or solvents is/are present in an amount sufficient to maintain analyte solubility and enable enzymatic hydrolysis. In some embodiments, the water miscible organic solvent comprises acetonitrile. In some embodiments, the acetonitrile is present in an amount of about 60% (vol/vol). In some embodiments, the water miscible organic solvent comprises methanol. In some embodiments, the methanol is present in an amount of about 60% (vol/vol). In some embodiments, the water miscible organic solvents comprise a mixture of acetonitrile and methanol. In some embodiments, the acetonitrile plus the methanol are present in a total amount of about 60% (vol/vol).

In some embodiments, the addition of water miscible solvent or solvents in amounts of about 10% (vol/vol or wt/vol) or more to the stock solution of enzyme effectively inhibits bacterial growth in that solution. For example, it is well known that 10% or more ethanol or methanol is an effective preservative for pharmaceutical sterile solutions (USP 11^{th} ed.). While the enzyme stock solution is provided as “sterile” from filtration or heat treatment, if the solution is opened in a non-sterile environment and a small portion is removed to use in hydrolysis of a glucuronide conjugate, the sterility of the stock solution is mitigated leading to bacterial growth and degradation of the enzyme per se. The presence of methanol or ethanol or other water miscible solvents at or in excess of 10% will inhibit bacterial growth thereby maintaining the sterility of the stock solution.

In some embodiments, the addition of water miscible solvent or solvents in excess of 10% (vol/vol or wt/vol) to the stock solution of enzyme effectively inhibits adsorption of target analyte to the walls of test container when the enzyme mix is introduced to the matrix containing analyte during sample analysis. The use of silanized glassware has been a recommended protocol to combat adsorptive losses of compounds, like THCA (11-nor-9-carboxy-Δ⁹-tetrandyrocannabinol or THC-acid), which have physicochemical properties that encourage adherence to glass and plastic surfaces (Clarke's Analytical Forensic Toxicology, 2^{nd} ed.). In a similar way, the presence of 10% or more ethanol or methanol can eliminate active sites on the test container surface and inhibit analyte adsorption. This in turn minimizes reduced analyte recovery and preserves integrity of analytical results.

In some embodiments, the present disclosure provides a method of hydrolyzing a drug-glucuronide conjugate comprising contacting the drug-glucuronide conjugate with an enzyme in the presence of water miscible organic solvent or solvents.

In some embodiments, the present disclosure provides a method of determining a concentration of a drug in a urine sample, the method comprising obtaining a urine sample containing a drug-glucuronide conjugate; contacting the urine sample with an enzyme configured to cleave the drug-glucuronide conjugate to form a solution comprising the drug; and determining a concentration of the drug in the solution.

These and other embodiments of the present disclosure are disclosed in further detail herein below.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various solvent composition values specified in this application, unless expressly indicated otherwise, are stated as approximations as though the minimum and maximum values of composition within the stated ranges were both preceded by the word “about.” Also, the disclosure of ranges is intended as a continuous range including every composition value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the composition values presented herein and in all instances such ratios, ranges, and ranges of ratios represent various embodiments of the present invention.

### I. Selected Methods of Cleaving Drug-Glucuronide Conjugates

The present disclosure provides methods for cleaving a drug-glucuronide conjugate. In some embodiments, the method comprises enzymatically cleaving the drug-glucuronide conjugate, optionally at or near room temperature.

In some embodiments, the method comprises obtaining a urine sample containing a drug-glucuronide conjugate and contacting the urine sample with an enzyme configured to cleave the drug-glucuronide conjugate. In some embodiments, the step of contacting the urine sample is performed at or near room temperature, for example at no more than about 65° C., at no more than about 60° C., at no more than about 55° C., at no more than about 50° C., at no more than about 45° C., at no more than about 40° C., at no more than about 35° C., at no more than about 30° C., at no more than about 26° C., at no more than about 25° C., at no more than about 20° C., at no more than about 15° C., or at no more than about 10° C. In some embodiments, the step of contacting the urine sample is performed at room temperature (e.g., ambient temperature). In some embodiments, the step of contacting the urine sample is performed at a temperature of about 20° C. to about 30° C.

The drug can be any compound that forms conjugates with glucuronic acid or a derivative thereof, including a metabolite of the ingested drug or the ingested drug itself. In some embodiments, the drug is excreted at least in part as a conjugate with glucuronic acid or a derivative thereof (i.e., a “drug-glucuronide conjugate”). In some embodiments, the drug is a hydrophobic drug. The term “hydrophobic drug” as used herein refers to a drug that is more readily soluble in an organic solvent than in water. In some embodiments, the drug is an opioid. In some embodiments, the drug is a benzodiazepine. In some embodiments, the drug is a cannabinoid such as THC, dimethylheptylpyran (“DHMP”) or parahexyl, or a metabolite thereof such as 11-hydroxytetrahydrocannabinol (“11-OH-THC”) or tetrahydrocannabinolic acid (“THCA”).

In some embodiments, the enzyme configured to cleave the drug-glucuronide conjugate is a glucuronidase. In some embodiments, the glucuronidase comprises, consists essentially of, or consists of a recombinant glucuronidase. In some embodiments, the recombinant glucuronidase is capable of hydrolyzing drug-glucuronide conjugates having a planar drug (e.g., a benzodiazepine) more rapidly than drug-glucuronide conjugates having a relatively less planar drug (e.g., opiates, norbuprenorphine, etc.). In some embodiments, the recombinant glucuronidase is IMCSzyme™ (Integrated Micro-Chromatography Systems, Columbia, S.C.). In some embodiments, the glucuronidase comprises, consists essentially of, or consists of a naturally derived glucuronidase (e.g., abalone, snails, bacterial). In some embodiments, the naturally derived glucuronidase is Red Abalone beta-glucuronidase (Kura Biotec, Inglewood, Calif.).

In some embodiments, the step of contacting the urine sample with an enzyme configured to cleave the drug-glucuronide conjugate is allowed to react to completion. In some such embodiments, the step of contacting the urine sample with an enzyme configured to cleave the drug-glucuronide conjugate is performed for no more than about one hour, for example for no more than about 60 minutes, for no more than about 55 minutes, for no more than about 50 minutes, for no more than about 45 minutes, for no more than about 40 minutes, for no more than about 35 minutes, for no more than about 30 minutes, for no more than about 25 minutes, for no more than about 20 minutes, for no more than about 15 minutes, for no more than about 10 minutes, for no more than about 5 minutes, for no more than about 4 minutes, for no more than about 3 minutes, for no more than about 2 minutes, or for no more than about 1 minute.

In some embodiments, the step of contacting the urine sample with an enzyme configured to cleave the drug-glucuronide conjugate includes adding a solvent matrix before or concurrently with the enzyme. In some embodiments, the solvent matrix maintains solubility (e.g., substantial or complete solubility) of the drug-glucuronide conjugate, the drug (e.g., unconjugated drug in the urine sample and/or drug that has been cleaved from the glucuronide conjugate), and/or the enzyme. In some embodiments, the solvent matrix maintains solubility (e.g., substantial or complete solubility) of at least two of: (i) the drug-glucuronide conjugate, (ii) the drug (e.g., unconjugated drug in the urine sample and/or drug that has been cleaved from the glucuronide conjugate), and (iii) the enzyme. In some embodiments, the solvent matrix maintains solubility (e.g., substantial or complete solubility) of all three of: (i) the drug-glucuronide conjugate, (ii) the drug (e.g., unconjugated drug in the urine sample and/or drug that has been cleaved from the glucuronide conjugate), and (iii) the enzyme. In some embodiments, the drug is a hydrophobic drug.

In some embodiments, the solvent matrix comprises an organic solvent, optionally a water-miscible organic solvent. In some embodiments, the water-miscible organic solvent comprises, consists essentially of, or consists of methanol, ethanol, acetonitrile, dimethyl formamide (“DMF”), dimethyl sulfoxide (“DMSO”), or a combination thereof. In some embodiments, the solvent matrix additionally comprises water. In other embodiments, the solvent matrix does not include water. In some embodiments, the solvent matrix comprises about 20% to about 80%, by volume, of the organic solvent, for example about 20%, about 25%, about 30%, about 35%, about 40%, about 45%, about 50%, about 55%, about 60%, about 65%, about 70%, about 75%, or about 80%, by volume, of the organic solvent. In some embodiments, the organic solvent represents about 20% to about 80% of the total liquid volume after addition to the urine sample, for example about 20% to about 80%, by volume, of the organic solvent, for example about 20%, about 25%, about 30%, about 35%, about 40%, about 45%, about 50%, about 55%, about 60%, about 65%, about 70%, about 75%, or about 80% of the total liquid volume after addition to the urine sample.

In some embodiments, the present disclosure provides a method of hydrolyzing a drug-glucuronide conjugate comprising contacting the drug-glucuronide conjugate with an enzyme in the presence of water miscible organic solvent. In some embodiments, the drug-glucuronide conjugate is in aqueous solution. In some embodiments, the water miscible organic solvent is present in an amount of at least about 20% (vol/vol %). In some embodiments, the water miscible organic solvent is present in an amount of at least about 40% (vol/vol %). In some embodiments, the e water miscible organic solvent is present in an amount of at least about 60% (vol/vol %). In some embodiments, the step of contacting the drug-glucuronide conjugate with the enzyme occurs at a temperature of no more than about 65° C.

In some embodiments, the step of contacting the drug-glucuronide conjugate with the enzyme occurs at a temperature of no more than about 55° C. In some embodiments, the step of contacting the drug-glucuronide conjugate with the enzyme occurs at a temperature of about 19° C. to about 25° C. In some embodiments, the solvent comprises at least one of methanol, ethanol, dimethyl formamide (DMF), and dimethyl sulfoxide (DMSO). In some embodiments, the solvent is inert and polar. In some embodiments, the enzyme comprises a recombinant glucuronidase. In some embodiments, the recombinant glucuronidase is IMCSzyme™. In some embodiments, the enzyme comprises a naturally derived glucuronidase. In some embodiments, the naturally derived glucuronidase is Red Abalone beta-glucuronidase. In some embodiments, the naturally derived glucuronidase is E. coli derived beta-glucuronidase. In some embodiments, the naturally derived glucuronidase is Helix pomatia beta-glucuronidase.

### II. Selected Methods of Determining Drug Concentrations

In some embodiments, the present disclosure provides methods of determining a concentration of a drug in a urine sample, the method comprising obtaining a urine sample containing a drug-glucuronide conjugate, contacting the urine sample with an enzyme configured to cleave the drug-glucuronide conjugate, to form a solution comprising the drug, and determining a concentration of the drug in the solution. In some embodiments, the method further comprises extrapolating the concentration of the drug in the solution to determine a concentration of the drug in the urine sample.

In some embodiments, the step of contacting the urine sample is performed at or near room temperature, for example at no more than about 65° C., at no more than about 60° C., at no more than about 55° C., at no more than about 50° C., at no more than about 45° C., at no more than about 40° C., at no more than about 35° C., at no more than about 30° C., at no more than about 26° C., at no more than about 25° C., at no more than about 20° C., at no more than about 15° C., or at no more than about 10° C. In some embodiments, the step of contacting the urine sample is performed at room temperature (e.g., ambient temperature). In some embodiments, the step of contacting the urine sample is performed at a temperature of about 20° C. to about 30° C.

The drug can be any compound that forms conjugates with glucuronic acid or a derivative thereof, including a metabolite of the ingested drug or the ingested drug itself. In some embodiments, the drug is excreted at least in part as a drug-glucuronide conjugate. In some embodiments, the drug is a hydrophobic drug. In some embodiments, the drug is an opioid. In some embodiments, the drug is a benzodiazepine. In some embodiments, the drug is a cannabinoid such as THC, DHMP or parahexyl, or a metabolite thereof such as 11-OH-THC or THCA. In some embodiments, the drug is a synthetic cannabinoid or a metabolite thereof such as the 5-pentanoic acid derivative of JWH-018 (JWH-018 pentanoic acid), or of UR-144 (UR-144 pentanoic acid).

In some embodiments, the enzyme configured to cleave the drug-glucuronide conjugate is a glucuronidase. In some embodiments, the glucuronidase comprises, consists essentially of, or consists of a recombinant glucuronidase. In some embodiments, the recombinant glucuronidase is capable of hydrolyzing drug-glucuronide conjugates having a planar drug (e.g., a benzodiazepine) more rapidly than drug-glucuronide conjugates having a relatively less planar drug (e.g., opiates, norbuprenorphine, etc.). In some embodiments, the recombinant glucuronidase is IMCSzyme™ (Integrated Micro-Chromatography Systems, Columbia, S.C.). In some embodiments, the glucuronidase comprises, consists essentially of, or consists of a naturally derived glucuronidase. In some embodiments, the naturally derived glucuronidase is Red Abalone beta-glucuronidase (Kura Biotec, Inglewood, Calif.).

In some embodiments, the step of contacting the urine sample with an enzyme configured to cleave the drug-glucuronide conjugate is allowed to react to completion. In some such embodiments, the step of contacting the urine sample with an enzyme configured to cleave the drug-glucuronide conjugate is performed for no more than about one hour, for example for no more than about 60 minutes, for no more than about 55 minutes, for no more than about 50 minutes, for no more than about 45 minutes, for no more than about 40 minutes, for no more than about 35 minutes, for no more than about 30 minutes, for no more than about 25 minutes, for no more than about 20 minutes, for no more than about 15 minutes, for no more than about 10 minutes, for no more than about 5 minutes, for no more than about 4 minutes, for no more than about 3 minutes, for no more than about 2 minutes, or for no more than about 1 minute.

In some embodiments, the step of contacting the urine sample with an enzyme configured to cleave the drug-glucuronide conjugate includes adding a solvent matrix before or concurrently with the enzyme. In some embodiments, the solvent matrix maintains solubility (e.g., substantial or complete solubility) of the drug-glucuronide conjugate, the drug (e.g., unconjugated drug in the urine sample and/or drug that has been cleaved from the glucuronide conjugate), and/or the enzyme. In some embodiments, the solvent matrix maintains solubility (e.g., substantial or complete solubility) of at least two of: (i) the drug-glucuronide conjugate, (ii) the drug (e.g., unconjugated drug in the urine sample and/or drug that has been cleaved from the glucuronide conjugate), and (iii) the enzyme. In some embodiments, the solvent matrix maintains solubility (e.g., substantial or complete solubility) of all three of: (i) the drug-glucuronide conjugate, (ii) the drug (e.g., unconjugated drug in the urine sample and/or drug that has been cleaved from the glucuronide conjugate), and (iii) the enzyme. In some embodiments, the drug is a hydrophobic drug.

In some embodiments, the solvent matrix comprises an organic solvent, optionally a water-miscible organic solvent. In some embodiments, the water-miscible organic solvent comprises, consists essentially of, or consists of methanol, ethanol, acetonitrile, dimethyl formamide (“DMF”), dimethyl sulfoxide (“DMSO”), or a combination thereof. In some embodiments, the solvent matrix additionally comprises water. In other embodiments, the solvent matrix does not include water. In some embodiments, the solvent matrix comprises about 20% to about 80%, by volume, of the organic solvent, for example about 20%, about 25%, about 30%, about 35%, about 40%, about 45%, about 50%, about 55%, about 60%, about 65%, about 70%, about 75%, or about 80%, by volume, of the organic solvent. In some embodiments, the organic solvent represents about 20% to about 80% of the total liquid volume after addition to the urine sample, for example about 20% to about 80%, by volume, of the organic solvent, for example about 20%, about 25%, about 30%, about 35%, about 40%, about 45%, about 50%, about 55%, about 60%, about 65%, about 70%, about 75%, or about 80% of the total liquid volume after addition to the urine sample.

The step of determining the concentration of the drug in the solution can include any known analytical technique useful for the particular drug of interest. For example, the step of determining the concentration of the drug in the solution may include subjecting a portion of the solution to chromatography (e.g., HPLC, GC, etc.), mass spectroscopy, fluorescence spectrophotometry or spectroscopy, Ultraviolet-Visible (UV-Vis) spectrophotometry, infra-red spectrophotometry, enzyme techniques, immunoassay technology or a combination thereof (e.g., GC-MS-MS, LC-MS-MS, LC-UV-Vis etc.). One of skill in the art will readily be able to select a suitable analytical technique for quantifying the amount of the drug in the solution.

In some embodiments, the method further comprises extrapolating the concentration of the drug in the solution to determine a concentration of the drug in the urine sample. The step of extrapolating the concentration of the drug in the solution to determine a concentration of the drug in the urine sample can include any method known to those of skill in the art. For example, the step of extrapolating may include mathematically converting the concentration of the drug in the solution based on one or more dilution steps performed on the urine sample and/or on the solution prior to the step of determining the concentration of the drug in the solution.

In some embodiments, the present disclosure provides a method of determining a concentration of a drug in a urine sample, the method comprising obtaining a urine sample containing a drug-glucuronide conjugate; contacting the urine sample with an enzyme configured to cleave the drug-glucuronide conjugate to form a solution comprising the drug; and determining a concentration of the drug in the solution. In some embodiments, the method further comprises extrapolating the concentration of the drug in the solution to determine a concentration of the drug in the urine sample. In some embodiments, the method further comprises diluting the urine sample before determining the concentration of the drug in the solution. In some embodiments, the method further comprises adding a water miscible organic solvent to the urine sample. In some embodiments, the step of adding the solvent further includes adding water to the urine sample. In some embodiments, the solvent is present in an amount of at least about 20% (vol/vol %). In some embodiments, the solvent is present in an amount of at least about 40% (vol/vol %). In some embodiments, the solvent is present in an amount of at least about 60% (vol/vol %). In some embodiments, the step of contacting the drug-glucuronide conjugate with the enzyme occurs at a temperature of no more than about 65° C. In some embodiments, the step of contacting the drug-glucuronide conjugate with the enzyme occurs at a temperature of no more than about 55° C. In some embodiments, the step of contacting the drug-glucuronide conjugate with the enzyme occurs at a temperature of about 19° C. to about 25° C. In some embodiments, the solvent comprises methanol. In some embodiments, the solvent comprises at least one of ethanol, acetonitrile, dimethyl formamide (DMF), and dimethyl sulfoxide (DMSO). In some embodiments, the solvent is inert and polar. In some embodiments, the enzyme comprises a recombinant glucuronidase. In some embodiments, the recombinant glucuronidase is IMCSzyme™. In some embodiments, the enzyme comprises a naturally derived glucuronidase. In some embodiments, the naturally derived glucuronidase is Red Abalone beta-glucuronidase (Kura Biotec, Inglewood, Calif.)

### EXAMPLES

### Example 1

### Enzymatic Hydrolysis of Benzodiazepine-Glucuronide Conjugates and Buprenorphine Conjugates with a Recombinant Enzyme, IMCSzyme™

### Preparation of IS

To a 10 mL volumetric flask add 6 mL of deionized (DI) water. Add 100 μL each of Oxazepam D5 (100 μg/mL), Temazepam D5 (100 μg/mL), Alphahydroxyalprazolam D5 (100 μg/mL), Nordiazepam D5 (100 μg/mL), and 500 μL of 7-Aminoclonazepam D4 (100 μg/mL). Quantum satis (QS) to the line with DI water, cap and invert several times to mix well. Concentrations of the internal standard (IS) compounds are 1000 ng/mL for Oxazepam D5, Temazepam D5, Alphahydroxyalprazolam D5, nordiazepam D5, and 5000 ng/mL for 7-aminoclonazepam D4.

### Control Prep

In a 10 mL volumetric flask add 1 mL NHU. Add 250 μL of Oxazepam Glucuronide/Lorazepam Glucuronide/Temazepam Glucuronide working solution (100 μg/mL) unadjusted to allow quantitation of Oxazepam, Lorazepam and Temazepam at 1548, 1614, and 1557 ng/mL, respectively, post-hydrolysis. QS to line with NHU.

### Sample Prep

Pipet 250 μL sample into a 7504 autosampler vial. Then pipet 50 μL of Benzodiazepine IS into vial. Pipet 12.5 μL of enzyme stock (>50000 U) into vial (this will yield 625 U, minimum, in final 352.5 μL volume). Pipet 40 μL of 0.2M Sodium Phosphate buffer, pH 6.8, and wait 5, 10, 15, and 20 minutes. Vortex and move to instrument. For heated hydrolysis, vortex and move to oven at 55° C. Wait 5, 15, 30, 45, and 60 minutes and then move to instrument.

### Preparation of Buprenorphine Mastermix Solution (Enzyme Stock+Internal Standard+Buffer)

To a 100 mL volumetric flask add 50 mL of 0.2M Sodium Phosphate buffer, pH 6.8. Then add 5 mL of IMCS enzyme stock (>50000 U) and 50 μL each of Buprenorphine D4 (1 mg/mL) and Norbuprenorphine D3 (1 mg/mL). QS to the line with 0.2M Sodium Phosphate buffer, pH 6.8, cap and invert several times to mix well. Final enzyme concentration is 2500 U. Final IS concentration is 500 ng/mL.

### Control Preparation

To a 10 mL volumetric flask add 1 mL NHU. Add 75 μL of Buprenorphine Glucuronide/Norbuprenorphine Glucuronide working solution (100 μg/mL) adjusted to allow quantitation of Buprenorphine and Norbuprenorphine at 750 ng/mL post-hydrolysis. QS to line with NHU.

### Sample Preparation

Pipet 100 μL sample into 750 μL autosampler vial. Pipet 400 μL of prepared Buprenorphine mastermix into vial (this will yield 1000 U in final 500 μL volume). Vortex and move to oven at 55 or 65° C. Wait 15, 30, 45, and 60 minutes. Move to instrument. For 0 minutes time point, skip oven and move immediately to instrument.

As shown in Table 1, the recombinant enzyme rapidly cleaves Benzodiazepine and Buprenorphine Glucuronides while requiring some time and heat to cleave Norbuprenorphine Glucuronide.

TABLE 1
IMCSzyme ™ Hydrolysis Efficiency for Glucuronides for Common Benzodiazepines and
Buprenorphine and Norbuprenorphine

| Time (mins) at | Mean % Hydrolysis | | |
|---|---|---|---|
| 55° C. | Oxazepam | Lorazepam | Temazepam |
| 5 | 88.8 | 89.3 | 79.0 |
| 15 | 94.2 | 92.8 | 87.8 |
| 30 | 93.0 | 92.9 | 90.5 |
| 45 | 96.9 | 98.5 | 93.5 |
| 60 | 95.7 | 94.6 | 93.9 |
| Time (mins) at | | | |
| Room | | | |
| Temperature | Oxazepam | Lorazepam | Temazepam |
| 0 | 90.5 | 91.8 | 80.3 |
| 5 | 94.3 | 94.1 | 81.7 |
| 10 | 92.0 | 91.6 | 80.7 |
| 15 | 91.9 | 93.1 | 81.9 |

| | Mean % Hydrolysis | | | |
|---|---|---|---|---|
| | 55° C. | | 65° C. | |
| | Buprenorphine | Norbuprenorphine | Buprenorphine | Norbuprenorphine |
| Time (mins) | Glucuronide | Glucuronide | Glucuronide | Glucuronide |
| 0 | 105.6 | 11.6 | 105.6 | 36.8 |
| 15 | 104.6 | 81.7 | 110.9 | 92.0 |
| 30 | 102.6 | 95.2 | 107.5 | 100.9 |
| 45 | 99.8 | 98.2 | 106.4 | 102.2 |
| 60 | 101.4 | 99.4 | 106.9 | 101.7 |

### Example 2

### Enzymatic Hydrolysis of Opiate-Glucuronide Conjugates with a Recombinant Enzyme, IMCSzyme™

### Preparation of Opiate Mastermix Solution (Enzyme Stock+Internal Standard+Buffer)

To a 100 mL volumetric flask add 50 mL of 0.2M Sodium Phosphate buffer, pH 6.8. Then add 21.75 mL of IMCS enzyme stock (>50000 U) and 91 μL each of Noroxycodone D3 (1 mg/mL) and Norhydrocodone D3 (1 mg/mL) and 45.5 μL each of Oxycodone D3, Oxymorphone D3, Morphine D3, Codeine D3, Hydrocodone D6, and Hydromorphone D3. QS to the line with 0.2M Sodium Phosphate buffer, pH 6.8, cap and invert several times to mix well. Final enzyme concentration is 10875 U. Final IS concentration is 455/910 ng/mL.

### Control Preparation

To a 10 mL volumetric flask add 1 mL NHU. Add 2504 of Morphine-3-Glucuronide/Morphine-6-Glucuronide/Hydromorphone Glucuronide/Codeine Glucuronide/Oxymorphone Glucuronide working solution (100 μg/mL) unadjusted to allow quantitation of Morphine, Codeine, Hydromorphone and Oxymorphone at 1549, 1577, 1549, and 1581 ng/mL, respectively, post-hydrolysis. QS to line with NHU.

### Sample Preparation

Pipet 125 μL sample into 750 μL autosampler vial. Pipet 275 μL of prepared Opiate mastermix into vial (this will yield 3000 U in final 400 μL volume). Vortex and move to oven at 55 or 65° C. Wait 15, 30, 45, and 60 minutes. Move to instrument. For 0 minutes time point, skip oven and move immediately to instrument.

As shown in Table 2, the recombinant enzyme efficiently cleaves most opiate-glucuronide conjugates within one hour at 55° C. or 65° C.

TABLE 2
IMCSzyme ™ Hydrolysis Efficiency for Glucuronides of Common Opiates

| | Mean % Hydrolysis | | | | |
|---|---|---|---|---|---|
| | Morphine-3- | Morphine-6- | Oxymorphone | Hydromorphone | Codeine |
| | Glucuronide | Glucuronide | Glucuronide | Glucuronide | Glucuronide |
| Time (mins) | | | | | |
| at 55° C. | | | | | |
| 0 | 99.5 | 40.4 | 43.6 | 58.5 | 15.7 |
| 15 | 99.1 | 67.9 | 71.7 | 80.5 | 40.2 |
| 30 | 99.1 | 83.6 | 90.5 | 91.1 | 65.6 |
| 45 | 97.4 | 95.9 | 97.0 | 93.3 | 76.6 |
| 60 | 99.9 | 91.7 | 101.2 | 93.8 | 81.8 |
| Time (mins) | | | | | |
| at 65° C. | | | | | |
| 0 | 99.5 | 40.4 | 43.6 | 58.5 | 15.7 |
| 15 | 95.9 | 71.2 | 76.2 | 77.1 | 42.7 |
| 30 | 95.8 | 88.3 | 89.7 | 89.4 | 64.1 |
| 45 | 97.9 | 98.5 | 95.6 | 91.0 | 75.5 |
| 60 | 97.6 | 97.9 | 100.0 | 91.5 | 79.8 |

### Example 3

### Enzymatic Hydrolysis of Cannabinoid-Glucuronide Conjugates with a Recombinant Enzyme, IMCSzyme™

### Preparation of Enzyme Stock Solution

To a 10 mL volumetric flask add 5 mL of 0.2M Sodium Phosphate buffer pH 7.5. Then add 1.0 mL of IMCS enzyme. QS to the line with 0.2M phosphate buffer, cap and invert several times to mix well. Final enzyme concentration is 5000 U.

### Preparation of Enzyme Stock Solution with Organic

To a 10 mL volumetric flask add 6 mL of 50/50 ACN/MeOH. Add 254 of THCA D9 100 μg/mL. Then add 1.0 mL of IMCS enzyme stock (>5000 U). QS to the line with 0.2M sodium phosphate buffer. Final enzyme concentration is 5000 U.

### Preparation of IS

In a 10 mL volumetric flask add 6 mL of 50/50 ACN/MeOH. Add 25 μL of THCA D9 (100 μg/mL). QS to the line with DI water, cap and invert several times to mix well. Concentration of IS will be 250 ng/mL.

### Control Preparation

To a 10 mL volumetric flask add 1 mL ACN. Add 18.5 μL of 10 μg/mL THCA Glucuronide working solution adjusted to allow quantitation of THCA at 18.5 ng/mL post hydrolysis. QS to line with NHU.

### Sample Preparation without Organic

Pipet 50 μL sample into a 750 μL autosampler vial. Pipet 50 μL of prepared enzyme into vial (this will yield 500 U in final 500 μL volume) and wait 5, 10, 15, and 20 minutes. Then pipet 400 μL of THCA D9 IS into the vial. Vortex and move to instrument.

### Sample Preparation with Organic

Pipet 50 μL sample into a 750 μL, autosampler vial. Then pipet 450 μL, of Enzyme Stock Solution With Organic (see above) into vial. Wait 5, 10, 15, and 20 minutes. Vortex and move to instrument.

Table 3 shows the hydrolysis of THCA-glucuronide conjugate at room temperature, 55° C. and 65° C. as a function of order of addition of the enzyme and the organic solvent (acetonitrile/methanol).

TABLE 3
IMCSzyme ™ Hydrolysis Efficiency for THCA Glucuronide using a Commercial Control at 18.5 ng/mL (post-hydrolysis) in the Presence of Aqueous Buffer and Water Miscible Organic Media
ICMS Enzyme Hydrolysis of THCA Glucuronide at 5 min 55, 65° C. and Room Temperature

| THCA Glucuronide | Expected | Actual | | THCA Glucuronide | Expected | Actual | |
|---|---|---|---|---|---|---|---|
| 55° C. | Conc. | Conc. | % Dev. | 65° C. | Conc. | Conc. | % Dev. |
| 5 min 01 | 18.5 | 19.9 | 7.04 | 5 min 01 | 18.5 | 19.7 | 6.09 |
| 5 min 02 | 18.5 | 20.9 | 11.48 | 5 min 02 | 18.5 | 19.8 | 6.57 |
| 5 min 03 | 18.5 | 20.1 | 7.96 | 5 min 03 | 18.5 | 19.7 | 6.09 |
| 5 min 04 | 18.5 | 21.6 | 14.35 | 5 min 04 | 18.5 | 22.6 | 18.14 |
| 5 min 05 | 18.5 | 21.3 | 13.15 | 5 min 05 | 18.5 | 21.2 | 12.74 |

| | | | |
|---|---|---|---|
| Average % Accuracy | 89.20 | Average % Accuracy | 90.07 |

| THCA Glucuronide. | Expected | Actual | | THCA Glucuronide | Expected | Actual | |
|---|---|---|---|---|---|---|---|
| 5 minutes Room Temp | Conc. | Conc. | % Dev | 10 minutes Room Temp. | Conc. | Conc. | % Dev |
| 5 min 01 | 18.5 | 17.1 | −8.19 | 10 min 01 | 18.5 | 17.4 | −6.32 |
| 5 min 02 | 18.5 | 14.5 | −27.59 | 10 min 02 | 18.5 | 21.9 | 15.53 |
| 5 min 03 | 18.5 | 17.8 | −3.93 | 10 min 03 | 18.5 | 16.8 | −10.12 |
| 5 min 04 | 18.5 | 18.3 | −1.09 | 10 min 04 | 18.5 | 18.5 | 0.00 |
| 5 min 05 | 18.5 | 16.8 | −10.12 | 10 min 05 | 18.5 | 17.2 | −7.56 |

| | | | |
|---|---|---|---|
| Average % Accuracy | 110.20 | Average % Accuracy | 101.70 |

The data in Table 3 indicate that the recombinant enzyme hydrolysis of THCA-glucuronide conjugate is complete at room temperature in the time it takes to fill the 96-well plate (e.g. ˜15 min using robotics). For all practical purposes, hydrolysis of this planar molecule is complete upon addition of the enzyme to the sample. In contrast, more 3 dimensional molecules linked to glucuronic acid take more time and heat to cleave in the presence of the IMCSzyme™ (see Table 2, Morphine-6-glucuronide, Codeine-6-glucuronide, etc.).

Table 4 shows the impact of organic media on the hydrolysis of THCA glucuronide using IMCSzyme™. Sample preparation was identical to that for Table 3.

TABLE 4
Hydrolysis of THCA-glucuronide with and without Water
Miscible Organic Media.

| | Mean % Hydrolysis |
|---|---|
| | THCA Glucuronide |
| Enzyme Added and | |
| Incubated, then Organic | |
| Media Added | |
| Time (mins) at Temp (° C.) | |
| 5 min at 55° C. | 89.2 |
| 5 min at 65° C. | 90.1 |
| Enzyme and Organic Media | |
| added at same time | |
| Time (mins) at Room | |
| Temperature | |
| 5 min at Room Temp | 110.2 |
| 10 min at Room Temp | 101.7 |
| 15 min at Room Temp | 104.9 |

Table 4 shows the relative data from experiments wherein hydrolysis was tested in a) aqueous buffer alone and b) aqueous buffer with acetonitrile/methanol added using a commercial control sample that yields 18.5 ng/mL post hydrolysis. The similarity of these data around the expected result of 18.5 ng/mL indicates that the added organic solvent does not inhibit or denature the enzyme in any way in the course of the experiment.

### Example 4

### Enzymatic Hydrolysis of Cannabinoid-Glucuronide Conjugates with a Naturally Derived Enzyme, Red Abalone Beta-Glucuronidase (Kura Biotec, Inglewood, Calif.)

### Preparation of Enzyme Stock Solution

To a 10 mL volumetric flask add 5 mL of DI water. Then add 1.0 mL of Kura Biotec enzyme. QS to the line with DI water, cap and invert several times to mix well. Final enzyme concentration is 5000 U.

### Preparation of Enzyme Stock Solution with Organic

In a 10 mL volumetric flask add 6 mL of 50/50 ACN/MeOH. Add 25 μL of THCA D9 100 μg/mL. Then add 1.0 mL of IMCS enzyme. QS to the line with DI water. Final enzyme concentration is 5000 U.

### Preparation of IS

To a 10 mL volumetric flask add 6 mL of 50/50 ACN/MeOH. Add 25 μL of THCA D9 (100 μg/mL). QS to the line with DI water, cap and invert several times to mix well. Concentration of IS will be 250 ng/mL.

### Control Preparation

To a 10 mL volumetric flask add 1 mL ACN. Add 18.5 μL of 10 μg/mL THCA Glucuronide working solution adjusted to allow quantitation of THCA at 18.5 ng/mL post hydrolysis. QS to line with NHU.

### Sample Preparation without Organic

Pipet 50 μL sample into a 750 μL autosampler vial. Pipet 50 μL of prepared enzyme into vial (this will yield 500 U in final 500 μL volume). Vortex and heat 15, 30, 45, and 60 minutes. Then pipet 400 μL of THCA D9 IS into vial. Vortex and move to instrument.

### Sample Preparation with Organic

Pipet 50 μL sample into a 750 μL autosampler vial. Then pipet 450 μL of Enzyme Stock Solution With Organic (see above) into vial. Vortex and heat 15, 30, 45, and 60 minutes. Vortex and move to instrument.

TABLE 5
Red Abalone beta-Glucuronidase Hydrolysis Efficiency for THCA
Glucuronide using a Commercial Control at 18.5 ng/mL (post hydrolysis).

| | | | | Enzyme + |
|---|---|---|---|---|
| | | | | Organic |
| | Temp | No Enzyme | Enzyme | Solvent(s) |
| Time (min) | (° C.) | (% Hydrolysis) | (% Hydrolysis) | (% Hydrolysis) |
| 15 | 65° C. | 0.03 | 104 | 121 |
| 30 | 65° C. | 0.03 | 97 | 130 |
| 45 | 65° C. | 0.03 | 102 | 134 |
| 60 | 65° C. | 0.03 | 101 | 136 |

The data in Table 5 shows that in the absence of enzyme at elevated temperature there is no hydrolysis under the conditions of the experiment. In the presence of enzyme, the THCA glucuronide is hydrolyzed completely by 15 min. In the presence of the enzyme and 60% water miscible solvent, the hydrolysis proceeds to 100% as early as 15 min demonstrating that this enzyme is not denatured or otherwise adversely impacted by the presence of this water miscible organic solvent mixture.

### Further Examples

### Example 5

A method of hydrolyzing a drug-glucuronide conjugate comprising contacting the drug-glucuronide conjugate with an enzyme in the presence of a water miscible organic solvent.

### Example 6

The method of Example 5, wherein the drug-glucuronide conjugate is in aqueous solution containing a water miscible organic solvent.

### Example 7

The method of Example 5 or Example 6, wherein the water miscible organic solvent is present in an amount of at least about 10% (vol/vol %).

### Example 8

The method of any one of Examples 5-7, wherein the water miscible organic solvent is present in an amount of at least about 20% (vol/vol %).

### Example 9

The method of any one of Examples 5-8, wherein the water miscible organic solvent is present in an amount of at least about 40% (vol/vol %).

### Example 10

The method of any one of Examples 5-9, wherein the water miscible organic solvent is present in an amount of at least about 60% (vol/vol %).

### Example 11

The method of any one of Examples 5-10, wherein the step of contacting the drug-glucuronide conjugate with the enzyme occurs at a temperature of no more than about 65° C.

### Example 12

The method of any one of Examples 5-11, wherein the step of contacting the drug-glucuronide conjugate with the enzyme occurs at a temperature of no more than about 55° C.

### Example 13

The method of any one of Examples 5-12, wherein the step of contacting the drug-glucuronide conjugate with the enzyme occurs at a temperature of about 19° C. to about 25° C.

### Example 14

The method of any one of Examples 5-13, wherein the solvent comprises methanol.

### Example 15

The method of any one of Examples 5-14, wherein the solvent comprises ethanol.

### Example 16

The method of any one of Examples 5-15, wherein the solvent comprises acetonitrile.

### Example 17

The method of any one of Examples 5-16, wherein the solvent comprises dimethyl formamide (DMF).

### Example 18

The method of any one of Examples 5-17, wherein the solvent comprises dimethyl sulfoxide (DMSO).

### Example 19

The method of any one of Examples 5-18, wherein the solvent comprises a polar, water miscible solvent.

### Example 20

The method of any one of Examples 5-19, wherein the solvent is inert, polar and water miscible.

### Example 21

The method of any one of Examples 5-20, wherein the water miscible solvent comprises 2 or more of: methanol, ethanol, acetonitrile, dimethylformamide (DMF), dimethyl sulfoxide (DMSO).

### Example 22

The method of any one of Examples 5-21, wherein the enzyme comprises a recombinant glucuronidase.

### Example 23

The method of Example 22, wherein the recombinant glucuronidase is IMCSzyme™.

### Example 24

The method of any one of Examples 5-23, wherein the enzyme comprises a naturally derived glucuronidase.

### Example 25

The method of Example 24, wherein the naturally derived glucuronidase is Red Abalone beta-glucuronidase.

### Example 26

The method of any one of Examples 5-25, wherein the water miscible organic solvent prevents bacterial growth in a solution of the enzyme.

### Example 27

The method of any one of Examples 5-26, wherein the contacting occurs in a test container, and wherein the water miscible organic solvent is present in an amount sufficient to substantially prevent the drug-glucuronide conjugate and/or a hydrolyzed portion thereof from adhering to a wall of the test container.

### Example 28

The method of Example 27, wherein the enzyme is provided as a solution comprising at least about 10% (vol/vol or wt/vol) of the water miscible organic solvent.

What is claimed is:

1. A method of determining a concentration of a drug in a urine sample, the method comprising:
obtaining a urine sample containing a drug-glucuronide conjugate;
contacting the urine sample with an enzyme configured to cleave the drug-glucuronide conjugate to form a solution comprising the drug; and
determining a concentration of the drug in the solution.

2. The method of claim 1 further comprising extrapolating the concentration of the drug in the solution to determine a concentration of the drug in the urine sample.

3. The method of claim 1 further comprising diluting the urine sample before determining the concentration of the drug in the solution.

4. The method of claim 1 further comprising adding a water miscible organic solvent to the urine sample.

5. The method of claim 1, wherein the step of adding the solvent further includes adding water to the urine sample.

6. The method of claim 1, wherein the solvent is present in an amount of at least about 20% (vol/vol %).

7. The method of claim 1, wherein the solvent is present in an amount of at least about 40% (vol/vol %).

8. The method of claim 1, wherein the organic solvent is present in an amount of at least about 60% (vol/vol %).

9. The method of claim 1, wherein the step of contacting the drug-glucuronide conjugate with the enzyme occurs at a temperature of no more than about 65° C.

10. The method of claim 1, wherein the step of contacting the drug-glucuronide conjugate with the enzyme occurs at a temperature of no more than about 55° C.

11. The method of claim 1, wherein the step of contacting the drug-glucuronide conjugate with the enzyme occurs at a temperature of about 19° C. to about 25° C.

12. The method of claim 1, wherein the solvent comprises methanol.

13. The method of claim 1, wherein the solvent comprises ethanol.

14. The method of claim 1, wherein the solvent comprises acetonitrile.

15. The method of claim 1, wherein the solvent comprises dimethyl formamide (DMF).

16. The method of claim 1, wherein the solvent comprises dimethyl sulfoxide (DMSO).

17. The method of claim 1, wherein the solvent is inert and polar.

18. The method of claim 1, wherein the “solvent” is inert and polar and is a combination of 2 or more of methanol, ethanol, acetonitrile, dimethylformamide (DMF), dimethyl sulfoxide (DMSO).

19. The method of claim 1, wherein the enzyme comprises a recombinant glucuronidase.

20. The method of claim 19, wherein the recombinant glucuronidase is IMCSzyme™.

21. The method of claim 1, wherein the enzyme comprises a naturally derived glucuronidase.

22. The method of claim 21, wherein the naturally derived glucuronidase is Red Abalone beta-glucuronidase.

23. The method of claim 1, wherein the water miscible organic solvent prevents bacterial growth in a solution of the enzyme.

24. The method of claim 1, wherein the contacting occurs in a test container, and wherein the water miscible organic solvent is present in an amount sufficient to substantially prevent the drug-glucuronide conjugate and/or the drug from adhering to a wall of the test container.

25. The method of claim 1, wherein the enzyme is provided as a solution comprising at least about 10% (vol/vol or wt/vol) of the water miscible organic solvent.
